(19) United States
(12) Patent Application Publication
(10) Pub. No.: US 2008/0255475 A1
(43) Pub. Date: Oct. 16, 2008

(54) GUIDEWIRE-ASSISTED CATHETER PLACEMENT SYSTEM

(75) Inventors: John D. KONDROSKY, Caledon (CA); Abtihal RAJI-KUBBA, Phoenix, AZ (US)

Correspondence Address: Rutan & Tucker, LLP., 611 ANTON BLVD, SUITE 1400, COSTA MESA, CA, 92626, US

(73) Assignee: C. R. Bard, Inc., Murray Hill, NJ (US)

(21) Appl. No.: 12/104,253

(22) Filed: Apr. 16, 2008

Related U.S. Application Data
(60) Provisional application No. 60/923,636, filed on Apr. 16, 2007.

(51) Int. Cl. A61M 25/09

(52) U.S. Cl. 600/585; 604/164.13

(57) ABSTRACT

A catheter assembly for providing precise placement of a catheter distal end at a desired location within the patient vasculature is disclosed. In one embodiment, the catheter assembly comprises a catheter including an elongate body that defines a proximal end, a distal end, and a lumen extending therebetween. A guidewire is also included and is configured for being received within the lumen of the catheter and for guiding the catheter through the patient vasculature. The guidewire includes a plurality of depth markings along at least a portion of a length of the guidewire. The depth markings indicate a distance between a distal end of the guidewire and an insertion site through which the guidewire passes into the patient vasculature. The guidewire further includes a modified tip configured for assisting in guidewire advancement through the vasculature, and a proximal end orientation feature that indicates the orientation of the modified tip.

### CROSS-REFERENCE TO RELATED APPLICATION

This application claims the benefit of U.S. Provisional Patent Application No. 60/923,636, filed Apr. 16, 2007, and entitled “Catheter Placement System,” which is incorporated herein by reference in its entirety.

### BRIEF SUMMARY

The present invention has been developed in response to the above and other needs in the art. Briefly summarized, embodiments of the present invention are directed to a catheter assembly for providing intravascular access to a patient is disclosed. The catheter assembly is configured for precise placement of the catheter distal end at a desired location within the patient vasculature.

In one embodiment, the catheter assembly comprises a catheter including an elongate body that defines a proximal end, a distal end, and a lumen extending therebetween. A guidewire is also included and is configured for being received within the lumen of the catheter and for guiding the catheter through a vasculature of the patient.

The guidewire in one embodiment includes a plurality of depth markings along at least a portion of a length of the guidewire. The depth markings indicate a distance between a distal end of the guidewire and an insertion site through which the guidewire passes into the patient vasculature. Thus, when the distal end of the guidewire has been navigated to a desired location in the patient vasculature, the precise distance between the distal end and the insertion site can be readily ascertained by reading the depth marking at the insertion site. The place of the catheter can use this “depth” distance to then trim the catheter to the appropriate length before inserting into the patient. The catheter is then slid over the guidewire into the patient vasculature until the distal end of the catheter arrives at the desired location. The guidewire is then removed, and the catheter secured.

The guidewire in one embodiment further includes a modified tip at the distal end thereof that is configured for assisting in advancement of the guidewire through the vasculature. A compliant tip and a j-tip are examples of modified tips that may be employed. An orientation feature is also disposed at the proximal end of the guidewire that indicates the orientation of the modified tip. In this way, a placer of the catheter can readily determine the orientation of the tip of the guidewire within the patient by observing the external orientation feature at the guidewire proximal end.

These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof that are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a perspective view of a catheter assembly configured in accordance with one example embodiment of the present invention;

FIG. 2 is a perspective view of a guidewire included in the catheter assembly of FIG. 1;

FIG. 3 is a side view of the guidewire of FIG. 2, showing various features thereof according to one example embodiment;

FIG. 4 is a side view of the guidewire of FIG. 2, showing various features thereof according to another example embodiment;

FIG. 5 is a side view of a catheter included in the catheter assembly of FIG. 1;

FIGS. 6A and 6B depict various details regarding the insertion of the guidewire of FIG. 2 into a patient according to one possible technique;

FIGS. 7A and 7B depict various details regarding the insertion of the guidewire of FIG. 2 into a patient according to another possible technique;

FIGS. 8A and 8B are side and cross sectional views, respectively, of a guidewire including an orientation feature according to one example embodiment;

FIGS. 9A-10B are side and cross sectional views of additional examples of guidewire orientation features according to example embodiments; and

FIG. 11 is a side view of a distal portion of a guidewire forming a J-tip according to yet another example embodiment.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the invention, and are not limiting of the present invention nor are they necessarily drawn to scale.

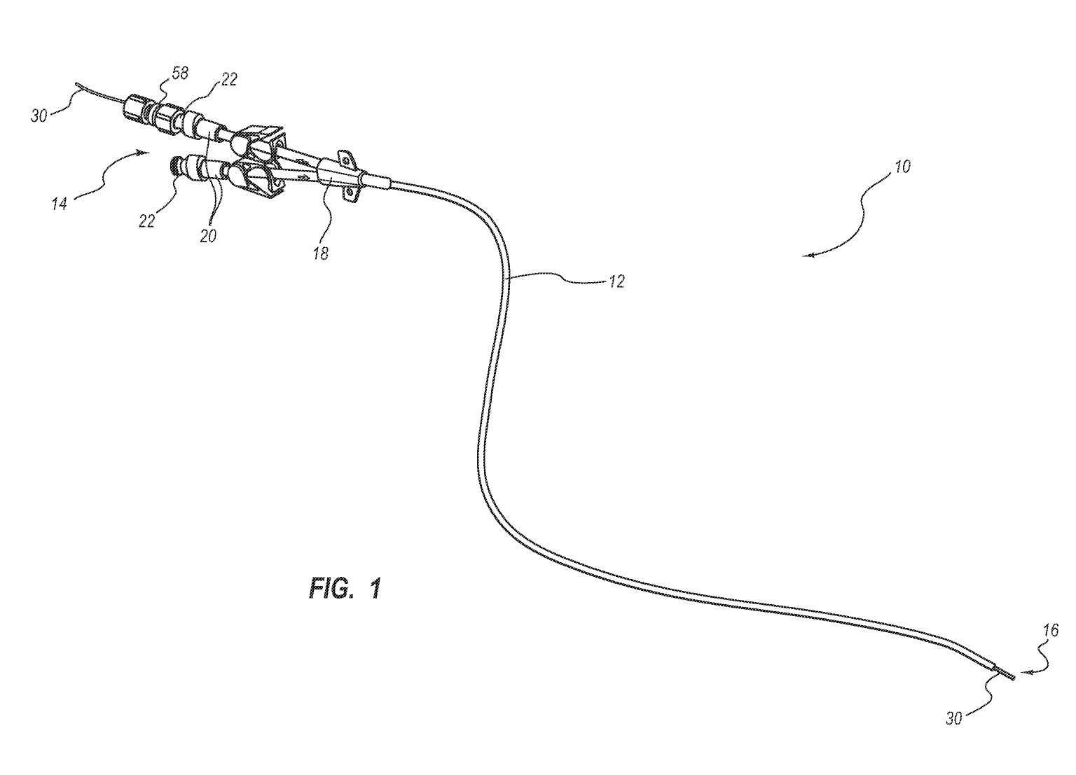

FIGS. 1-11 depict various features of embodiments of the present invention, which embodiments are generally directed to a catheter assembly configured for accurate placement within the vasculature of a patient.

Reference is first made to FIG. 1, which depicts a catheter assembly, generally designated at 10, configured in accordance with one example embodiment of the present invention. In detail, the catheter assembly 10 includes a catheter 12 defined by an elongate, tubular body that defines a lumen extending from a distal end 16 toward a proximal end 14 of the assembly. A hub 18 is included at the proximal end of the catheter 12, and extension legs 20 extend proximally from the hub. Each extension leg 20 includes a connector 22 for enabling connectivity with fluid delivery or aspiration components.

Note that the particular configuration of the components shown in FIG. 1, including the hub and extension legs may vary from what is described herein. For example, the number of extension legs may be more or less than two. Or, the number of lumens defined by the catheter may be more than one, for instance. Such variations from what is described herein are contemplated as residing within the claims of the present invention.

The catheter assembly further includes a guidewire 30 employed in assisting the placement of the catheter 12 in the vasculature of a patient. As shown in FIG. 1, the guidewire 30 passes through the catheter 12 and one of the extension legs 20 so as to extend between the proximal and distal ends 14, 16 of the catheter assembly 10.

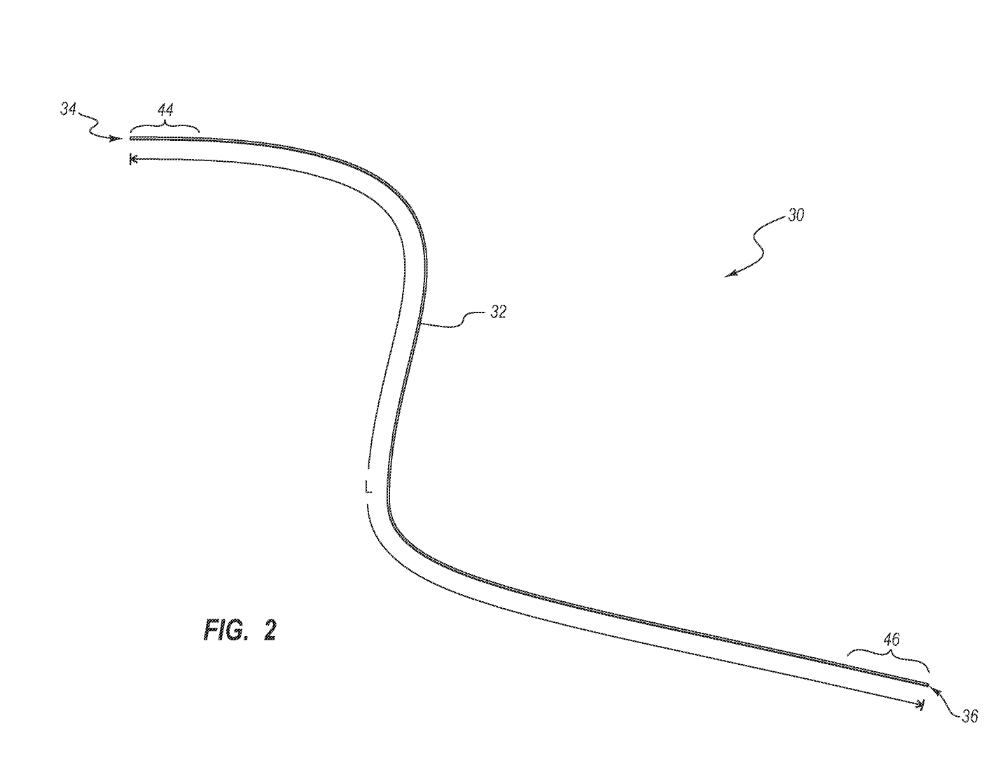

Reference is now made to FIG. 2 in describing various details regarding the guidewire 30. In particular, the guidewire 30 includes an elongate, tubular body 32 having a length L so as to define a proximal end 34 and a distal end 36. Additionally, a proximal region 44 is defined adjacent the proximal end 34, while a distal region 46 is defined adjacent the distal end 36. The body 32 here has a circular cross section, though it is appreciated that it can be formed in any number of shapes and sizes. The guidewire body 32 can include one or more of various materials, including stainless steel, nitinol, plastic, etc.

Note that, for purposes of the present disclosure, the term “guidewire” is construed herein to include any structure at least partially disposed within a vasculature of a patient and configured for reception by a lumen of a catheter or other suitable device to facilitate advancement of the catheter or other device into and/or within the vasculature of the patient.

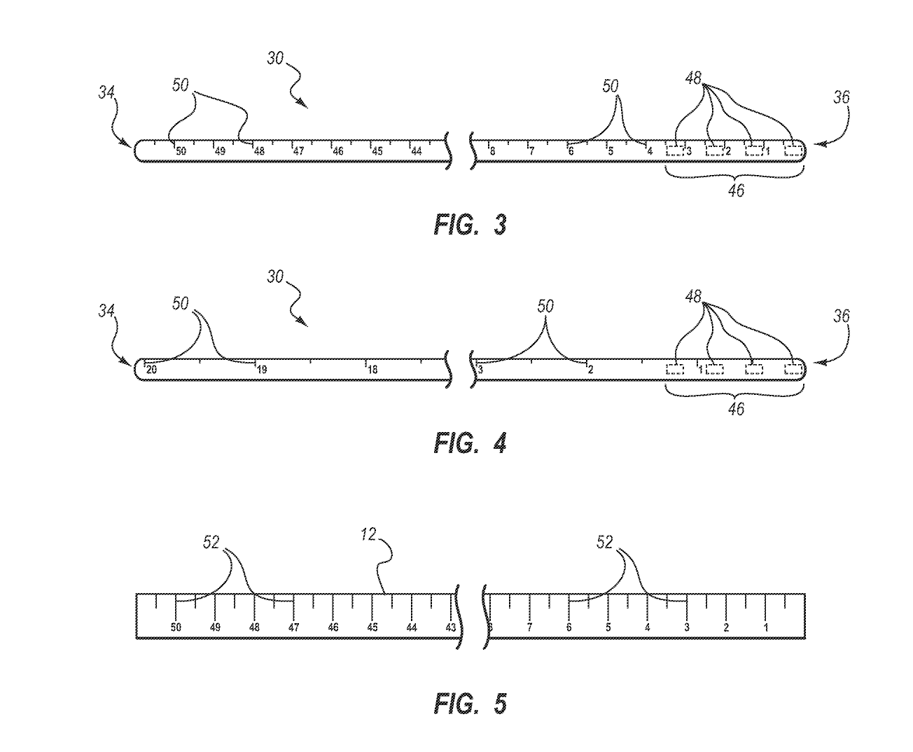

Reference is now made to FIG. 3 in describing various aspects of the guidewire 30, according to one example embodiment. As shown in FIG. 3, the guidewire 30 includes one or more magnetic elements 48 disposed within the guidewire at the distal region 46. Though shown here at plural elements, the magnetic elements 48 may include a singular structure. Indeed, in one embodiment, the distal region 46 of the guidewire 30 can be at least partially composed of a magnetic material. In the present embodiment, a plurality of magnetic elements 48 is incorporated into the distal region 48 up to the distal end 36. In another embodiment, the magnetic element(s) 48 can be proximally offset from the guidewire distal end 36.

Generally the magnetic elements 48 may include any type or form of magnetic material, including both permanent magnetic materials and electromagnetic materials. For example, in the present embodiment, the magnetic elements 48 include a rare-earth magnet (e.g., samarium cobalt and/or neodymium iron boron). In another embodiment, the magnetic elements can include an AINiCO magnetic material, a plastic magnetic material (e.g., PANiCNQ), or a ceramic magnetic material, such as barium ferrite (BaO6Fe2O3) or strontium ferrite (SrO6Fe2O3) and iron oxide (Fe3O4). In yet another embodiment, the magnetic materials can include an electromagnetic material such as a solenoid, which generates a magnetic field upon application of an electric current.

In the present embodiment, the magnetic elements 48 exhibit an observable dipole so as to provide an indication of the position and/or orientation of the magnetic elements and, therefore, the position and/or orientation of the distal region 46 of the guidewire 30. In particular, the magnetic elements 48 produce a magnetic dipole that, when the guidewire 30 is disposed within the vasculature of a patient, is detectable from outside of the patient's body using detection technology (discussed in greater detail below) to indicate the position and/or orientation of the guidewire 30 within the patient's body.

Generally speaking, the poles of the magnetic elements 48 of the guidewire 30 can be positioned or oriented in any number of ways. For example, the dipole of the magnetic elements 48 can be oriented substantially parallel to the longitudinal axis of the guidewire or substantially perpendicular to the longitudinal axis. In addition, the north pole of the magnetic elements 48, if commonly aligned, can be positioned proximate the distal end 36 of the guidewire 30, thus orienting the south pole of the magnetic elements toward the proximal end 34.

In general, any type or form of detection system may be used to detect the dipole or other aspect of the magnetic element(s) 48 to provide an indication of the position and/or orientation of the distal end 36 of the guidewire 30 when in the vasculature of a patient. Non-limiting examples of suitable detection apparatus include the various detection devices disclosed in U.S. Pat. Nos. 5,879,297, 6,129,668, 6,216,028, and 6,263,230 to Haynor et al. (“the Haynor patents”), the entirety of each of which is incorporated, in its entirety, by this reference. For example, an exemplary detection apparatus may comprise a plurality of magnetic sensors oriented in a known direction to generate a set of signals based on the strength and direction of the magnetic field generated by the magnetic element(s) of the guidewire 30. A processor may then calculate an estimated position of the magnetic elements 48 in a three-dimensional space based on the predicted and actual magnetic field strength of the magnetic material derived from the set of signals generated by the magnetic sensors. It is also appreciated that in other embodiments, an ECG-based detector may be used to detect the position of the guidewire distal end 36 with respect to the SVC or other portion of the heart, as may be appreciated by one skilled in the art.

For example, the location and/or orientation of the magnetic element(s) 48 of the guidewire 30 can be calculated by comparing the difference between the predicted magnetic field strength and the actual measured magnetic field strength of the magnetic element(s). In certain embodiments, a display connected to the processor may display the position of the magnetic material of the guidewire 30 in a three-dimensional space. Accordingly, a detection apparatus, such as the exemplary detection apparatus described herein, may detect the magnetic field generated by the magnetic material of guidewire 30 positioned within a patient's body in order to determine the position and/or orientation of at least a portion of the guidewire, such as the distal end thereof.

Note that the present embodiment contemplates use of the guidewire 30 with a catheter, such as a central venous catheter (“CVC”), or peripherally-inserted central catheter (“PICC”) to help guide the catheter 12 (FIG. 1) into the superior vena cava (“SVC”) portion of the vasculature of a patient. However, it should be appreciated that the guidewire discussed herein can also be employed with other catheters or for directing the catheter to areas of vasculature other than the SVC. The embodiments described herein are therefore exemplary only.

As seen in FIG. 3, the guidewire 30 includes a plurality of numbered depth markings 50. The depth markings 50 represent a graduated scale indicating length along the guidewire body 32. Though accompanied here by numbers arranged in increasing numerical order from the distal end 36, the depth markings 50 can in other embodiments be accompanied by numbers in descending order from the distal end or by symbols, letters, or other indicia. The depth markings 50 of FIG. 3 are in centimeter graduations, while those shown in FIG. 4 are shown in inch graduations, though other increments are also possible. The depth markings 50 serve as a graduated scale indicating the distance along the guidewire from a point of reference, such as an insertion site where the guidewire enters the vasculature of the patient, to one of either the proximal or distal ends 34 or 36.

The depth markings 50 can be placed on the guidewire 30 in one or more of a variety of ways, including via physical or chemical etching, engraving, imprinting, etc. In one embodiment, the depth markings can be disposed on the guidewire 30 so as to be radiographically observable, if desired.

In greater detail, when the guidewire 30 has been placed such that its distal end 36 is located at a desired position within the patient vasculature, such as the SVC, the depth marking 50 closest to the insertion site can be consulted to determine the distance from the insertion site to the guidewire distal end. This immediately informs the placer of the catheter how long the catheter 12 must be in order to traverse the same path through the vasculature form the insertion site in order to disposed the distal end of the catheter at the desired position. This in turn provides enhanced catheter distal tip placement accuracy.

FIG. 5 shows that the catheter 12 can also include markings 52 that correspond with the depth markings of the guidewire 30, such as the depth markings 50 shown on the guidewire in FIG. 3. Use of a similar set of markings on the catheter 12 enables relative movement to occur between the catheter and the guidewire 30 during insertion of the catheter assembly into the patient vasculature, as will be described.

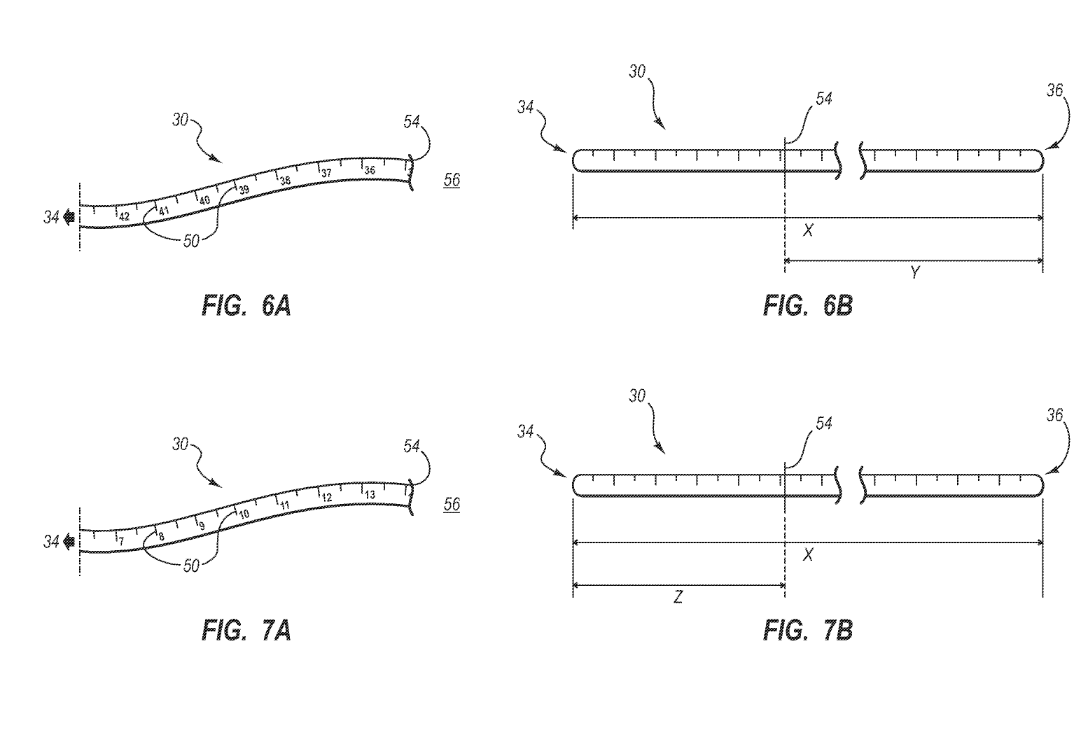

Reference is now made to FIGS. 6A and 6B in describing use of the guidewire 30 having depth markings 50 in inserting a catheter or similar device within the vasculature of a patient. Note that the method to be described below may include other steps or utilize additional components than what is described herein. In accordance with known techniques, a needle, cannula, or other device is used to pierce through the skin of a patient 56 at an insertion site 54 into a vein or artery, thereby establishing access to the vasculature of the patient. The guidewire 30, having depth markings 50 that ascend in order from the distal end 36, is inserted through the insertion site 54 and advanced along the vasculature while the position and advancement of the guidewire distal region 46 is monitored by an external magnetic detection device or other suitable detection apparatus.

The monitoring by the magnetic detection device confirms that the distal end 36 of the guidewire 30 arrives at a desired location within the vasculature of the patient, such as the SVC. Once the distal end 36 of the guidewire 30 is positioned at the desired location, the placer notes the depth marking 50 nearest the insertion site 54. From this depth marking, the placer is able to determine the length of catheter necessary to reach the desired location. For instance, FIG. 6B shows that if the total length of the guidewire 30 is “X,” and the guidewire has been advanced a distance “Y” into the patient vasculature, the placer will know to cut the catheter 12 (FIG. 1) to a length similar to “Y” such that the catheter will reach the desired location without having an excess amount of catheter tubing remaining outside of the patient.

Once it has been cut to proper length according to the calculation of the depth marking 50 above, the catheter 12 is advanced through the insertion site 54 and over the guidewire 30 until the distal end of the catheter has reached the desired position. This will correspond to the external portion of the catheter 12 being in the desired proximity to the insertion site 54 as desired by the placer. The placer then removes the guidewire 30 and secures the catheter 12.

In another example embodiment, shown in FIGS. 7A and 7B, a guidewire is used that includes depth markings 50 that ascend in order from the proximal end 34 of the guidewire. In this case, the same process as above is followed, with an exception: the placer first notes the depth marking 50 closest to the insertion site 54. The placer then subtracts an amount “Z” (FIG. 7B), representing the amount of guidewire 30 still external to the patient body, from “X,” representing the total length of the guidewire. This result gives the length the catheter 12 should be trimmed to in order to position the catheter within the vasculature with the distal end thereof at the desired location while the proximal end is externally positioned sufficiently close to the insertion site 54. Note that the catheter 12 may be proximally or distally trimmable. Advantageously, placement of the catheter 12 as described above results in reduced numbers of mal-positions and relatively more accurate placement of catheter distal tips at a desired location within the vasculature of the patient.

In one embodiment, the guidewire 30 can be pre-loaded within the lumen of the catheter 12 to form an assembly and inserted into the patient vasculature in this configuration. When inserted in this manner, the distal end 36 of the guidewire 30 corresponds to the distal end of the catheter as the assembly is advanced through the patient vasculature. Thus, when the guidewire distal region 46, including the magnetic elements 48, is detected using a magnetic detection device as being at the SVC or other desired position, the distal portion of the catheter 12 is also positioned thereat. Corresponding markers (i.e., the depth markers 50 and the markers 52) of the guidewire 30 and catheter 12 enable the distance from the insertion site 54 to the desired location to be readily ascertained. However, should an obstruction or area of difficult passage be encountered during advancement of the mated catheter 12 and guidewire 30 to the desired location, the distal end 36 of the guidewire can be temporarily advanced beyond the distal end of the catheter to enable the obstruction to be more easily traversed by the guidewire. Once the distal end of the guidewire has advanced past the obstruction to the desired location, the catheter 12 can be advanced relative to the guidewire until its distal end is also at the desired location. Again, because of the markings 52 disposed on the catheter 12 (FIG. 5) that correspond to the depth markings 50 of the guidewire 30, the placer will be able to readily determine when the distal ends of the catheter 12 and guidewire 30 are both at the desired location. The guidewire 30 can then be removed. In one embodiment, a securement device is used to selectively lock the guidewire 30 to the catheter such that unintended advancement of the guidewire relative to the catheter is prevented. FIG. 1 shows one such securement device at 58, implemented as a Touhy-Borst adapter and connected to a proximal end of the extension leg 22, though other securement devices may alternatively be employed, including a piece of tape or other adhesive component to secure the guidewire to the catheter.

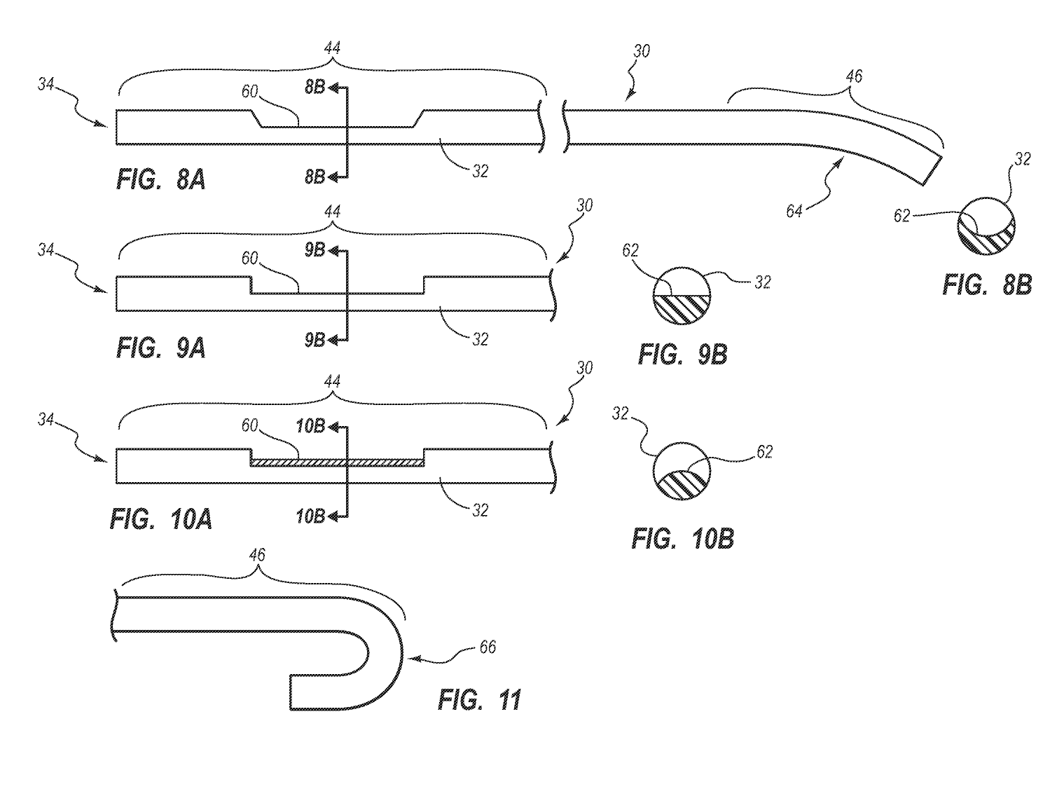

Reference is now made to FIGS. 8A and 8B, which depict various features of a guidewire in accordance with one example embodiment. In detail, the body 32 of the guidewire 30 at the proximal region 44 defines an orientation feature 60 for assisting the placer in determining the orientation of a feature at the distal region 46 of the guidewire. In the present embodiment, the orientation feature 60 is a concave cutout portion (FIG. 9B) extending longitudinally along a portion of the proximal region 44, while the feature at the guidewire distal region 46 is a modified tip, such as a compliant tip 64.

As shown, the compliant tip 64 includes a pre-curved portion defining a compliant bend. During advancement of the guidewire 30 through the patient vasculature, a placer can palpate or visually inspect the orientation feature 60 at the guidewire proximal region, which remains exterior to the patient. Knowing the orientation relationship between the orientation feature 60 and the compliant tip 64, the placer can easily determine the orientation of the compliant tip, thus assisting the placer in navigating the vasculature.

FIGS. 9A and 9B show the orientation feature 60 according to another example embodiment, defining a flat cutout, while FIGS. 10A and 10B show a convexly shaped orientation feature 60. Note that orientation features having one or more of a variety of shapes and configuration are contemplated here as residing within the claims of the present invention, including detents, beveled surfaces, depression, nubs, etc.

FIG. 11 depicts another example of a modified distal tip of the guidewire 30 according to another example embodiment. In particular, the modified tip defines a j-tip 66, which also assists in guiding the guidewire through the vasculature of the patient. Note here that many alternative tip configurations can be used on the guidewire distal region, including tips having angles or curvatures greater than or less than those shown in FIGS. 8A and 11, and such tips may be associated with one or more of a variety of orientation features.

The present invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. A guidewire for use in positioning a catheter within a vasculature of a patient, comprising:
an elongate body extending between a proximal end and a distal end; and
a plurality of depth markings disposed on the body of the guidewire, each depth marking indicating a distance with respect to one of the proximal and distal ends of the guidewire.

2. The guidewire as defined in claim 1, wherein the depth markings extend from the distal end to the proximal end of the guidewire.

3. The guidewire as defined in claim 1, wherein the depth markings include numerical graduations that ascend in order from the distal end.

4. The guidewire as defined in claim 1, wherein the depth markings include numerical graduations that ascend in order from the proximal end.

5. The guidewire as defined in claim 1, wherein the depth markings enable a distance between the distal end of the guidewire and an insertion site to be determined when the guidewire is inserted into the patient.

6. The guidewire as defined in claim 5, wherein the distance between the distal end of the guidewire and the insertion site is determined when the distal end of the guidewire is positioned at a desired location with the vasculature of the patient.

7. The guidewire as defined in claim 5, wherein the determined distance between the distal end of the guidewire and the insertion site enables a catheter to be trimmed prior to insertion into the vasculature of the patient.

8. The guidewire as defined in claim 7, wherein the guidewire is configured to be received by a lumen of the catheter during insertion thereof and is configured to be removed from the patient when a distal end of the catheter positioned at the desired location.

9. The guidewire as defined in claim 1, wherein the guidewire is configured such that a distal region of the guidewire is trackable within the vasculature of the patient using an ECG-based device.

10. The guidewire as defined in claim 1, further comprising at least one magnetic element disposed in a distal region of the guidewire, the at least one magnetic element enabling the distal portion of the guidewire to be tracked using a magnetic detection device disposed externally of the patient.

11. The guidewire as defined in claim 1, further comprising a modified distal tip at the distal end that facilitates advancement of the guidewire through the vasculature of the patient.

12. The guidewire as defined in claim 11, wherein the modified distal tip is a compliant distal tip.

13. The guidewire as defined in claim 11, further comprising an orientation feature at a proximal region of the guidewire, the orientation enabling the orientation of the modified tip to be determined.

14. The guidewire as defined in claim 1, wherein the depth markings are etched into the guidewire body.

15. The guidewire as defined in claim 1, wherein the depth markings are radiographically observable.

16. A method for placing a catheter in a vasculature of a patient, the method comprising:
accessing the vasculature at an insertion site;
via the insertion site, advancing a guidewire through the vasculature until a distal end of the guidewire is positioned at a desired location; and
using a plurality of depth markings defined on the guidewire, determining a distance between the insertion site and the distal end of the guidewire at the desired location.

17. The method for placing the catheter as defined in claim 16, further comprising:
trimming the catheter before insertion into the vasculature of the patient to a length equal to the distance determined between the insertion site and the distal end of the guidewire at the desired location.

18. The method for placing the catheter as defined in claim 17, further comprising:
inserting the catheter over the guidewire into the vasculature of the patient until a distal end of the catheter is at the desired location; and
removing the guidewire from the vasculature.

19. The method for placing the catheter as defined in claim 16, wherein a distal region of the guidewire includes at least one magnetic element, and wherein advancing the guidewire through the vasculature further comprises:
advancing the guidewire through the vasculature with assistance from a magnetic detection device disposed external to the patient, the magnetic detection device detecting a position of the at least one magnetic element of the distal region of the guidewire within the vasculature of the patient.

20. The method for placing the catheter as defined in claim 16, wherein advancing the guidewire through the vasculature further comprises:
advancing the guidewire through the vasculature with assistance from an ECG-based device disposed external to the patient, the ECG-based device detecting a position of a distal region of the guidewire within the vasculature of the patient.

21. A method for placing a catheter in a vasculature of a patient, the method comprising:
accessing the vasculature at an insertion site;
advancing a catheter assembly into the vasculature, the catheter assembly including a catheter defining a lumen, a guidewire disposed in the lumen of the catheter, a distal end of the guidewire including a modified distal tip configured for assisting in navigating the vasculature, a distal region of the guidewire including at least one magnetic element;
by a magnetic detection device, detecting a location of the distal region of the guidewire as it navigates the vasculature of the patient; and
advancing the distal end of the guidewire further ahead of a distal end of the catheter in the event an obstacle in the vasculature of the patient is encountered to enable the distal end of the catheter to arrive at a desired location.

22. The method for placing the catheter as defined in claim 21, wherein a plurality of depth markings are included on the guidewire to indicate a distance from the insertion site to the distal end of the guidewire, and wherein a plurality of markings are defined on the catheter that correspond to the depth markings of the guidewire, and wherein the method further comprises:
after advancing the distal end of the guidewire further ahead, using the depth markings on the guidewire and the markings on the catheter to align the distal end of the catheter with the distal end of the guidewire at the desired location.

23. The method for placing the catheter as defined in claim 21, wherein detecting a location of the distal region of the guidewire further comprises:
confirming when the distal end of the guidewire has arrived at the desired location.

24. The method for placing the catheter as defined in claim 21, wherein advancing the distal end further comprises:
unlatching a securement device that secures the catheter and the guidewire together before advancing the distal end of the guidewire further ahead of a distal end of the catheter.

25. A catheter assembly, comprising:
a catheter including an elongate body defining a proximal end, a distal end, and at least one lumen extending therebetween; and
a guidewire configured for being received within the lumen of the catheter and for guiding the catheter through a vasculature of a patient, the guidewire including:
a plurality of depth markings along at least a portion of a length of the guidewire, the depth markings indicating a distance between a distal end of the guidewire and an insertion site through which the guidewire passes into the vasculature of the patient; and
a modified tip at the distal end of the guidewire, the modified tip configured for assisting in navigating the guidewire through the vasculature of the patient.

26. The catheter assembly as defined in claim 25, further comprising:
an orientation feature disposed at a proximal region of the guidewire, the orientation feature corresponding to an orientation of the modified tip so as to enable a placer of the catheter assembly to determine the orientation of the modified tip within the vasculature of the patient.

27. The catheter assembly as defined in claim 26, wherein the orientation feature includes a cutout portion that longitudinally extends along a portion of the proximal region of the guidewire.

28. The catheter assembly as defined in claim 26, wherein the orientation feature includes one of a convex, straight, and concave cross sectional shapes.

29. The catheter assembly as defined in claim 25, wherein the depth markings are graduated in centimeters in ascending order from the distal end of the guidewire.

30. The catheter assembly as defined in claim 25, wherein the catheter is configured to slide over the guidewire for placement in the vasculature of the patient when the distal end of the guidewire is positioned at a desired location within the vasculature of the patient.

31. The catheter assembly as defined in claim 25, wherein the modified tip is compliant and is pre-curved.

32. The catheter assembly as defined in claim 25, wherein the modified tip includes a j-tip shape.

33. The catheter assembly as defined in claim 25, further including a securement device that releasably secures the catheter to the guidewire.

34. The catheter assembly as defined in claim 33, wherein the securement device is a Touhy-Borst adapter that is included proximally of a hub that is attached a proximal end of the catheter.

35. A guidewire for use in positioning a catheter within a vasculature of a patient, comprising:
an elongate body extending between a proximal end and a distal end;
a modified tip disposed at the distal end of the guidewire, the modified tip configured for assisting in navigating the guidewire through the vasculature of the patient; and
an orientation feature disposed at a proximal region of the guidewire, the orientation feature corresponding to an orientation of the modified tip so as to enable a placer of the catheter assembly to determine the orientation of the modified tip within the vasculature of the patient.

36. The guidewire as defined in claim 35, wherein the orientation feature includes a cutout portion that longitudinally extends along a portion of the proximal region of the guidewire.

37. The guidewire as defined in claim 36, wherein the cutout portion is a notch defined in the proximal region.

38. The guidewire as defined in claim 35, wherein the orientation feature includes one of a convex, straight, and concave cross sectional shapes.

39. The guidewire as defined in claim 35, wherein the modified tip is compliant and is pre-curved.

40. The guidewire as defined in claim 39, wherein the modified tip includes a j-tip shape.

41. The guidewire as defined in claim 35, further comprising a plurality of magnetic elements included in a distal portion of the guidewire, the magnetic elements enabling an external magnetic detection device to locate the distal portion of the guidewire within the vasculature of the patient.

42. The guidewire as defined in claim 35, further comprising a plurality of depth markings disposed on the body of the guidewire, each depth marking indicating a distance with respect to one of the proximal and distal ends of the guidewire.
